(19) United States
(12) Patent Application Publication
(10) Pub. No.: US 2025/0282700 A1
(43) Pub. Date: Sep. 11, 2025

(54) METHOD FOR PRODUCING TRIFLUOROETHYLENE

(71) Applicant: ARKEMA FRANCE, Colombes (FR)

(72) Inventors: Cédric LAVY, Pierre Benite Cedex (FR); Kevin HISLER, Pierre Benite Cedex (FR); Alexandre CAMBRODON, Pierre Benite Cedex (FR); Thierry LANNUZEL, Pierre Benite Cedex (FR)

(73) Assignee: ARKEMA FRANCE, Colombes (FR)

(21) Appl. No.: 18/861,873

(22) PCT Filed: May 3, 2023
(86) PCT No.: PCT/EP2023/061695

§ 371 Date: Oct. 30, 2024

(30) Foreign Application Priority Data
May 3, 2022 (FR) FR2204198

(51) Int. Cl. C07C 17/25

(52) U.S. Cl. CPC C07C 17/25

(57) ABSTRACT

The present invention relates to a process for producing trifluoroethylene in a reactor equipped with a fixed catalytic bed comprising a catalyst, said process comprising a step a) of reacting a composition A comprising chlorotrifluoroethylene with hydrogen in the presence of a catalyst and in the gas phase in order to produce a stream B comprising trifluoroethylene, characterized in that said composition A also comprises at least one of the additional compounds C1 chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane. The present invention also relates to a chlorotrifluoroethylene composition.

### TECHNICAL FIELD OF THE INVENTION

The present invention relates to a process for producing hydrofluoroolefins. In particular, the present invention relates to a process for producing trifluoroethylene (HFO-1123 or VF₃) by hydrogenolysis of chlorotrifluoroethylene. The present invention also relates to a composition comprising chlorotrifluoroethylene.

### TECHNOLOGICAL BACKGROUND OF THE INVENTION

Fluoroolefins, such as VF₃, are known and are used as monomers or comonomers for the manufacture of fluorocarbon polymers exhibiting noteworthy characteristics, in particular excellent chemical resistance and good thermal resistance.

Trifluoroethylene is a gas under standard conditions of pressure and temperature. The main risks associated with the use of this product relate to its flammability, its propensity for self-polymerization when it is not stabilized, its explosiveness due to its chemical instability and its supposed sensitivity to peroxidation, by analogy with other halogenated olefins. Trifluoroethylene exhibits the distinguishing feature of being extremely flammable, with a lower explosive limit (LEL) of approximately 10% and an upper explosive limit (UEL) of approximately 30%. The major hazard, however, is associated with the propensity of VF₃ to decompose violently and explosively under certain pressure conditions in the presence of an energy source, even in the absence of oxygen.

Given the main risks above, the synthesis and also the storage of VF₃ pose particular problems and impose strict safety rules throughout these processes. A known route for the preparation of trifluoroethylene uses, as starting materials, chlorotrifluoroethylene (CTFE) and hydrogen in the presence of a catalyst and in the gas phase.

A process for producing trifluoroethylene by hydrogenolysis of CTFE in the gas phase and in the presence of a catalyst based on a metal from group VIII at atmospheric pressure and at relatively low temperatures is known from WO2013/128102.

A process for producing trifluoroethylene is known from EP 2 993 213. This can be obtained by hydrogenolysis of chlorotrifluoroethylene or by thermal decomposition of chlorodifluoromethane and chlorofluoromethane. The production process involves carrying out a distillation step at a pressure of 10 barg and by which the trifluoroethylene is recovered by sidestream withdrawal. Carrying out a high-pressure distillation requires the installation of particular operating conditions due to the explosive nature of trifluoroethylene above 3 bara.

There thus exists a need to provide a simpler and safer process for producing trifluoroethylene while maintaining high yields and selectivities.

### SUMMARY OF THE INVENTION

According to a first aspect, the present invention provides a process for producing trifluoroethylene in a reactor equipped with a fixed catalytic bed comprising a catalyst, said process comprising a step a) of reacting a composition A comprising chlorotrifluoroethylene with hydrogen in the presence of a catalyst and in the gas phase in order to produce a stream B comprising trifluoroethylene, characterized in that said composition A also comprises at least one of the additional compounds C1 chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane.

Surprisingly, it was observed that the trifluoroethylene productivity was increased in the presence of the additional compounds C1. The presence of these compounds in a small amount in addition to chlorotrifluoroethylene makes it possible to improve the process for producing trifluoroethylene. The present invention demonstrates that it is not necessary to have highly pure chlorotrifluoroethylene in order to achieve high productivities. This makes it possible to simplify the production process and to limit the overall cost thereof.

According to a preferred embodiment, the total weight content of said at least one of the additional compounds C1 is less than 15% on the basis of the total weight of said composition A, preferably less than 10% on the basis of the total weight of said composition A, in particular less than 5% on the basis of the total weight of said composition A.

According to a preferred embodiment, said composition A comprises at least 80% by weight of chlorotrifluoroethylene on the basis of the total weight of said composition A, preferably at least 95% by weight of chlorotrifluoroethylene on the basis of the total weight of said composition A, in particular at least 90% by weight of chlorotrifluoroethylene on the basis of the total weight of said composition A.

According to a preferred embodiment, said composition A also comprises trifluoroethylene, preferably in a weight content of less than 5% on the basis of the total weight of said composition A.

According to a preferred embodiment, said composition A also comprises at least one of the additional compounds C2 chosen from the group consisting of 1,1,2-trifluoroethane, 1-chloro-1,1,2-trifluoroethane, 1-chloro-2,2-difluoroethylene, E/Z-1-chloro-1,2-difluoroethylene and 1-chloro-1,2,2-trifluoroethane.

According to a preferred embodiment, the weight content of said at least one of the additional compounds C2 is less than 5% on the basis of the total weight of said composition A.

In a preferred embodiment, the catalyst comprises palladium supported on α-alumina.

According to a preferred embodiment, the chlorotrifluoroethylene and the hydrogen are in the anhydrous form.

According to a preferred embodiment, said process comprises a step i′) of activating the catalyst, carried out prior to step a), by bringing said catalyst into contact with a gas stream comprising a reducing agent, an inert gas or a mixture thereof.

According to a preferred embodiment, during said step i′):
the temperature of the catalytic bed is increased from a temperature T1 to a temperature T2 that is greater than T1, with a temperature gradient of less than 0.5° C./min; or
the temperature of the catalytic bed is increased from a temperature T1 to a temperature T2 that is greater than T1, in increments.

According to a second aspect, the present invention provides a composition comprising at least 80% by weight of chlorotrifluoroethylene and at least one of the additional compounds chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane; the total weight content of said at least one of the additional compounds is less than 15% on the basis of the total weight of said composition.

### DETAILED DESCRIPTION OF THE INVENTION

The present invention relates to a process for producing trifluoroethylene comprising a step of hydrogenolysis reaction of chlorotrifluoroethylene (CTFE) with hydrogen in the gas phase and preferably in the presence of a catalyst.

According to a preferred embodiment, the process according to the invention described in the present patent application is carried out continuously.

According to a preferred embodiment, in the process described in the present patent application, the hydrogen is in the anhydrous form.

According to a preferred embodiment, in the process described in the present patent application, the chlorotrifluoroethylene is in the anhydrous form.

Carrying out the processes according to the invention in the presence of anhydrous hydrogen and/or chlorotrifluoroethylene makes it possible to effectively increase the lifetime of the catalyst and thus the overall productivity of the process. The term anhydrous refers to a weight content of water of less than 1000 ppm, advantageously 500 ppm, preferably of less than 200 ppm, in particular of less than 100 ppm, on the basis of the total weight of the compound under consideration.

### Catalyst

Preferably, the catalyst is based on a metal from columns 8 to 10 of the Periodic Table of the Elements. In particular, the catalyst is based on a metal selected from the group consisting of Pd, Pt, Rh, and Ru; preferably palladium.

Preferably, the catalyst is supported. The support is preferably selected from the group consisting of activated carbon, an aluminum-based support, calcium carbonate and graphite.

Preferably, the support is based on aluminum. In particular, the support is alumina. The alumina can be α-alumina. Preferably, the alumina comprises at least 90% of α-alumina. It was observed that the conversion of the hydrogenolysis reaction was improved when the alumina is an α-alumina. Thus, the catalyst is more particularly palladium supported on alumina, advantageously palladium supported on an alumina comprising at least 90% of α-alumina, preferably palladium supported on an α-alumina.

Preferably, the palladium represents from 0.01% to 5% by weight, on the basis of the total weight of the catalyst, preferably from 0.1% to 2% by weight, on the basis of the total weight of the catalyst.

In particular, said catalyst comprises from 0.01% to 5% by weight of palladium supported on alumina; preferably, the alumina comprises at least 90% of α-alumina; more preferentially, the alumina is an α-alumina.

### Activation of the Catalyst

Said catalyst is preferably activated before its use in step a). Preferably, the activation of the catalyst is carried out at high temperature and in the presence of a reducing agent, an inert gas or a mixture thereof.

According to a particular embodiment, the reducing agent is chosen from the group consisting of hydrogen, carbon monoxide, nitric monoxide, formaldehyde, C₁-C₆ alkanes and C₁-C₁₀ halohydrocarbons, or a mixture thereof; preferably hydrogen or a C₁-C₁₀ halohydrocarbon or a mixture thereof; in particular hydrogen, chlorotrifluoroethylene, trifluoroethylene, chlorotrifluoroethane, trifluoroethane or difluoroethane, or a mixture thereof.

The inert gas may be nitrogen or argon, preferably nitrogen.

Preferably, the activation of the catalyst is carried out at a temperature of between 100° C. and 400° C., in particular at a temperature of between 150° C. and 350° C. In particular, the activation of the catalyst is carried out at a temperature of between 100° C. and 400° C., in particular at a temperature of between 150° C. and 350° C., in the presence of hydrogen as reducing agent.

Preferably, during step i′), the temperature of the catalytic bed is increased from a temperature T1 to a temperature T2. In particular, during said step i′), the temperature of the catalytic bed is increased from a temperature T1 to a temperature T2 that is greater than T1, with a temperature gradient of less than 0.5° C./min. The temperature gradient applied makes it possible to prevent premature degradation of the catalyst and thus enables a better yield or better productivity of the hydrogenolysis reaction. In particular, the temperature is increased with a temperature gradient of less than 0.45° C./min or less than 0.40° C./min, or less than 0.35° C./min, or less than 0.30° C./min, or less than 0.25° C./min, or less than 0.20° C./min, or less than 0.15° C./min, or less than 0.10° C./min, or less than 0.05° C./min. The temperature T1 is the initial temperature of the activation step. This temperature T1 can be ambient temperature. Alternatively, the temperature T1 can be between 0° C. and 150° C., advantageously between 0° C. and 120° C., preferably between 0° C. and 100° C., more preferentially between 10° C. and 100° C., in particular between 20° C. and 100° C., more particularly between 20° C. and 75° C., favorably between 20° C. and 50° C. The temperature T2 represents the temperature to be reached during the activation phase. The temperature T2 is advantageously between 150° C. and 400° C., preferably between 155° C. and 375° C., more preferentially between 160° C. and 350° C., in particular between 165° C. and 325° C., more particularly between 170° C. and 320° C., favorably between 175° C. and 310° C., more favorably between 180° C. and 300° C. According to a preferred embodiment, the temperature T2 is advantageously between 185° C. and 290° C., preferably between 190° C. and 280° C., more preferentially between 195° C. and 270° C., in particular between 200° C. and 260° C. The temperature T2 may be maintained for from 5 min to 200 h, preferably from 10 min to 100 h, in particular from 15 min to 75 h, more particularly from 30 min to 50 h, favorably from 1 h to 25 h. The temperature T2 can be maintained for from 5 min to 24 h, preferably from 10 min to 20 h, in particular from 15 min to 15 h, more particularly from 30 min to 10 h, favorably from 1 h to 10 h.

The gas stream used during step i′) preferably does not comprise oxygen. Preferably, step i′) can be carried out with an amount of reducing agent of greater than 0.01 mol per gram of catalyst, preferably greater than 0.05 per gram of catalyst. In particular, step i′) can be carried out with an amount of reducing agent of between 0.01 and 10 mol per gram of catalyst, preferably between 0.05 and 5 mol per gram of catalyst.

According to another embodiment, during said step i′), the temperature of the catalytic bed is increased in increments from a temperature T1 to a temperature T2. The activation of the catalyst in increments makes it possible to increase the performance of the catalyst. Implementing increments makes it possible to prevent degradation of the catalyst. It has also been observed that the properties of the catalyst were additionally further improved if the rise in temperature between the increments is gradual and relatively slow compared to the usual conditions for activating a catalyst. Thus, preferably, in step i′), between two increments, the temperature is increased with a temperature gradient of less than 0.5° C./min. The temperature gradient applied between two increments makes it possible to prevent premature degradation of the catalyst and thus enables a better yield or better productivity of the hydrogenolysis reaction. In particular, the temperature is increased with a temperature gradient of less than 0.45° C./min or less than 0.40° C./min, or less than 0.35° C./min, or less than 0.30° C./min, or less than 0.25° C./min, or less than 0.20° C./min, or less than 0.15° C./min, or less than 0.10° C./min, or less than 0.05° C./min. The temperature T1 is the initial temperature of the activation step. This temperature T1 can be ambient temperature.

Alternatively, the temperature T1 can be between 0° C. and 150° C., advantageously between 0° C. and 120° C., preferably between 0° C. and 100° C., more preferentially between 10° C. and 100° C., in particular between 20° C. and 100° C., more particularly between 20° C. and 75° C., favorably between 20° C. and 50° C. The temperature T2 represents the temperature to be reached during the activation phase. The temperature T2 is advantageously between 150° C. and 400° C., preferably between 155° C. and 375° C., more preferentially between 160° C. and 350° C., in particular between 165° C. and 325° C., more particularly between 170° C. and 320° C., favorably between 175° C. and 310° C., more favorably between 180° C. and 300° C. According to a preferred embodiment, the temperature T2 is advantageously between 185° C. and 290° C., preferably between 190° C. and 280° C., more preferentially between 195° C. and 270° C., in particular between 200° C. and 260° C. The temperature T2 may be maintained for from 5 min to 200 h, preferably from 10 min to 100 h, in particular from 15 min to 75 h, more particularly from 30 min to 50 h, favorably from 1 h to 25 h. The temperature T2 can be maintained for from 5 min to 24 h, preferably from 10 min to 20 h, in particular from 15 min to 15 h, more particularly from 30 min to 10 h, favorably from 1 h to 10 h. Step i′) of activating the catalyst contains at least one increment between the temperature T1 and the temperature T2. Step i′) of activating the catalyst can comprise several increments between the temperature T1 and the temperature T2. Preferably, step i′) comprises at least one increment at a temperature T1a of between 90 and 120° C. The presence of an increment between 90° C. and 120° C. should be favored for increasing the lifetime of the catalyst. Step i′) can also comprise one or more increments between the temperature T1 and T1a and/or between the temperature T1a and T2. Preferably, each increment between the temperature T1 and the temperature T2 can last between 5 min and 200 h, preferably between 10 min and 100 h, in particular between 15 min and 75 h, more particularly between 30 min and 50 h. In particular, each increment between the temperature T1 and the temperature T2 can last between 5 min and 24 h, preferably between 10 min and 20 h, in particular between 15 min and 15 h, more particularly between 30 min and 10 h. In particular, the increment at the temperature T1a can last between 5 min and 200 h, preferably between 10 min and 100 h, in particular between 15 min and 75 h, more particularly between 30 min and 50 h. Favorably, the increment at the temperature T1a can last between 5 min and 24 h, preferably between 10 min and 20 h, in particular between 15 min and 15 h, more particularly between 30 min and 10 h.

The gas stream used during step i′) can be different over time. For example, the gas stream can comprise an inert gas between two increments and for example comprise a reducing agent between two other increments. In particular, the gas stream comprises an inert gas when step i′) is carried out between the temperature T1 and T1a, and the gas stream comprises a reducing agent, preferably hydrogen or C₁-C₁₀ halohydrocarbons as defined above, when step i′) is carried out between the temperature T1a and T2. Thus, the gas stream used during step i′) is modified during the increment carried out at the temperature T1a. Alternatively, the gas stream can comprise a reducing agent such as hydrogen or C₁-C₁₀ halohydrocarbons as defined above throughout the whole of step i′), optionally mixed with an inert gas such as nitrogen. It has been observed that the use of a reducing agent such as hydrogen or C₁-C₁₀ halohydrocarbons as defined above, optionally mixed with an inert gas such as nitrogen, during the temperature rise between the temperature T1a of said increment and the temperature T2, represents an additional advantage in terms of productivity. As mentioned above, the temperature T2 is maintained for a certain period of time. During this increment at the temperature T2, the gas stream can be modified. Thus, the gas stream, during the increment at the temperature T2, can comprise hydrogen or a C₁-C₁₀ halohydrocarbon as defined above; in particular, the gas stream, during the increment at the temperature T2, can comprise hydrogen, chlorotrifluoroethylene, trifluoroethane, trifluoroethylene, chlorotrifluoroethane or difluoroethane. Preferably, step i′) can be carried out with an amount of reducing agent of greater than 0.01 per gram of catalyst, preferably greater than 0.05 per gram of catalyst. In particular, step i′) can be carried out with an amount of reducing agent of between 0.01 and 10 mol per gram of catalyst, preferably between 0.05 and 5 mol per gram of catalyst.

According to another embodiment, step i′) of activating the catalyst comprises bringing said catalyst into contact with a gas stream that comprises chlorotrifluoroethylene and optionally hydrogen. It has been observed that chlorotrifluoroethylene (CTFE) made it possible to activate the catalyst, in particular when only hydrogen is also present. This makes it possible to improve the process for producing trifluoroethylene. Activation in the presence of CTFE makes it possible to activate the catalyst at lower temperatures and thus provides a less energy-consuming process. The process is further simplified because the reducing agent during the activation is also one of the reactants for the subsequent reaction. Preferably, in this embodiment, step i′) is carried out at a temperature T2′ of less than 100° C. This temperature T2′ can be reached from a temperature T1′ using a low temperature gradient. Thus, during said step i′), the temperature of the catalytic bed is increased from a temperature T1′ to a temperature T2′ that is greater than T1′; the temperature of the catalytic bed is preferably increased from a temperature T1′ to a temperature T2′ that is greater than T1′ with a temperature gradient of less than 0.5° C./min. The temperature gradient applied makes it possible to prevent premature degradation of the catalyst and thus enables a better yield or better productivity of the hydrogenolysis reaction. In particular, the temperature is increased with a temperature gradient of less than 0.45° C./min or less than 0.40° C./min, or less than 0.35° C./min, or less than 0.30° C./min, or less than 0.25° C./min, or less than 0.20° C./min, or less than 0.15° C./min, or less than 0.10° C./min, or less than 0.05° C./min.

Preferably, the temperature of the catalytic bed is increased by increasing the contact time, calculated as being the ratio of the volume, in liters, of catalyst to the total flow rate of said gas stream, in standard liters per second, at the inlet of the reactor. The contact time is between 1 and 60 seconds, preferably between 5 and 45 seconds, in particular between 10 and 30 seconds, more particularly between 15 and 25 seconds. The temperature T1′ can be between 0° C. and 50° C., advantageously between 10° C. and 50° C., preferably between 20° C. and 50° C. Preferably, the temperature T2′ is lower than the temperature T3 at which step a) is carried out. The temperature T3 is preferably between 100° C. and 180° C., more preferentially between 100° C. and 160° C., in particular between 120° C. and 160° C.

### Regeneration of the Catalyst

Said catalyst used in the present process can be regenerated. This regeneration step can be carried out in a temperature range of the catalytic bed of between 90° C. and 450° C. Preferably, the regeneration step is carried out in the presence of hydrogen. Carrying out the regeneration step makes it possible to improve the yield of the reaction compared with the initial yield before regeneration.

According to a preferred embodiment, the regeneration step can be carried out at a temperature of the catalytic bed of 90° C. to 300° C., preferably at a temperature of the catalytic bed of 90° C. to 250° C., more preferentially of 90° C. to 200° C., in particular of 90° C. to 175° C., more particularly at a temperature of the catalytic bed of 90° C. to 150° C. In particular, carrying out the regeneration step at a low temperature, for example of 90° C. to 200° C. or of 90° C. to 175° C. or of 90° C. to 150° C., makes possible the desorption of compounds which are harmful to the activity of the catalyst and/or makes it possible to limit phase transitions which modify the structure of the catalyst.

According to another preferred embodiment, the regeneration step can be carried out at a temperature of the catalytic bed of greater than 200° C., advantageously of greater than 230° C., preferably of greater than 250° C., in particular of greater than 300° C. The regeneration step can be carried out periodically as a function of the productivity or of the conversion obtained in step a). The regeneration step can be advantageously carried out at a temperature of the catalytic bed of between 200° C. and 300° C., preferably between 205° C. and 295° C., more preferentially between 210° C. and 290° C., in particular between 215° C. and 290° C., more particularly between 220° C. and 285° C., favorably between 225° C. and 280° C., more favorably between 230° C. and 280° C. Alternatively, the regeneration step can be carried out at a temperature of between 300° C. and 450° C., preferably between 300° C. and 400° C. The regenerated catalyst can be reused in step a) of the present process.

### Hydrogenolysis Reaction

The present invention comprises, as mentioned above, a step of hydrogenolysis reaction of a composition A comprising chlorotrifluoroethylene with hydrogen in order to produce a stream comprising trifluoroethylene. The hydrogenolysis step is carried out in the presence of a catalyst and in the gas phase. Preferably, the hydrogenolysis step is carried out in the presence of a preactivated catalyst and in the gas phase. The hydrogenolysis step consists in simultaneously introducing hydrogen, CTFE and optionally an inert gas, such as nitrogen, in the gas phase and in the presence of said catalyst, which is preferably activated.

Preferably, said step a) is carried out at a temperature of the fixed catalytic bed of between 50° C. and 250° C. Said step a) can be carried out at a temperature of the fixed catalytic bed of between 50° C. and 240° C., advantageously between 50° C. and 230° C., preferably between 50° C. and 220° C., more preferentially between 50° C. and 210° C., in particular between 50° C. and 200° C. Said step a) can also be carried out at a temperature of the fixed catalytic bed of between 60° C. and 250° C., advantageously between 70° C. and 250° C., preferably between 80° C. and 250° C., more preferentially between 90° C. and 250° C., in particular between 100° C. and 250° C., more particularly between 120° C. and 250° C. Said step a) can also be carried out at a temperature of the fixed catalytic bed of between 60° C. and 240° C., advantageously between 70° C. and 230° C., preferably between 80° C. and 220° C., more preferentially between 90° C. and 210° C., in particular between 100° C. and 200° C., more particularly between 100° C. and 180° C., favorably between 100° C. and 160° C., particularly preferably between 120° C. and 160° C.

The H₂/CTFE molar ratio is of between 0.5/1 and 2/1 and preferably of between 1/1 and 1.2/1. If an inert gas, such as nitrogen, is present in step a), the nitrogen/H₂ molar ratio is of between 0/1 and 2/1 and preferably of between 0/1 and 1/1.

Step a) is preferably carried out at a pressure of 0.05 MPa to 1.1 MPa, more preferentially of 0.05 MPa to 0.5 MPa, in particular at atmospheric pressure.

The contact time, calculated as being the ratio of the volume, in liters, of catalyst to the total flow rate of the gas mixture, in standard liters per second, at the inlet of the reactor, is of between 1 and 60 seconds, preferably between 5 and 45 seconds, in particular between 10 and 30 seconds, more particularly between 15 and 25 seconds.

According to the present invention, said composition A also comprises at least one of the additional compounds C₁ chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane.

Said composition A may comprise one or more of the additional compounds C1. Said composition A may comprise one, two, three, four, five or all of the additional compounds C1.

Advantageously, the total weight content of said at least one of the additional compounds C1 is less than 15% on the basis of the total weight of said composition A. Preferably, the total weight content of said at least one of the additional compounds C1 is less than 10%, more preferentially less than 5%, in particular less than 2%, more particularly less than 1%.

Advantageously, the total weight content of said at least one of the additional compounds C1 is greater than 1 ppm on the basis of the total weight of said composition A. Preferably, the total weight content of said at least one of the additional compounds C1 is greater than 5 ppm, more preferentially greater than 10 ppm, in particular greater than 20 ppm, more particularly greater than 50 ppm, favorably greater than 100 ppm on the basis of the total weight of said composition A.

According to a preferred embodiment, the composition A comprises 1,1,1-trifluoroethane and the total weight content of 1,1,1-trifluoroethane is less than 5000 ppm, advantageously less than 2500 ppm, preferably less than 1000 ppm, more preferentially less than 750 ppm on the basis of the total weight of said composition A. When it is contained in the composition, the total weight content of 1,1,1-trifluoroethane is greater than 1 ppm, advantageously greater than 5 ppm, preferably greater than 10 ppm, more preferentially greater than 20 ppm, in particular greater than 50 ppm, more particularly greater than 100 ppm on the basis of the total weight of said composition A.

According to a preferred embodiment, the composition A comprises 1,1,1,2-tetraluoroethane and the total weight content of 1,1,1,2-tetraluoroethane is less than 1000 ppm, advantageously less than 750 ppm, preferably less than 500 ppm, more preferentially less than 250 ppm, in particular less than 100 ppm on the basis of the total weight of said composition A. When it is contained in the composition, the total weight content of 1,1,1,2-tetraluoroethane is greater than 1 ppm, advantageously greater than 5 ppm, preferably greater than 10 ppm, more preferentially greater than 20 ppm on the basis of the total weight of said composition A.

According to a preferred embodiment, the composition A comprises hexafluorocyclobutene and the total weight content of hexafluorocyclobutene is less than 1%, advantageously less than 7500 ppm, preferably less than 5000 ppm, more preferentially less than 2500 ppm, in particular less than 1000 ppm on the basis of the total weight of said composition A. When it is contained in the composition, the total weight content of hexafluorocyclobutene is greater than 1 ppm, advantageously greater than 5 ppm, preferably greater than 10 ppm, more preferentially greater than 20 ppm, in particular greater than 50 ppm, more particularly greater than 100 ppm on the basis of the total weight of said composition A.

According to a preferred embodiment, the composition A comprises fluoroethane and the total weight content of fluoroethane is less than 100 ppm, advantageously less than 75 ppm, preferably less than 50 ppm, more preferentially less than 25 ppm, in particular less than 10 ppm on the basis of the total weight of said composition A. When it is contained in the composition, the total weight content of fluoroethane is greater than 0.1 ppm, advantageously greater than 0.5 ppm, preferably greater than 1 ppm on the basis of the total weight of said composition A.

According to a preferred embodiment, the composition A comprises 2-chloro-1,1,1-trifluoroethane and the total weight content of 2-chloro-1,1,1-trifluoroethane is less than 1%, advantageously less than 7500 ppm, preferably less than 5000 ppm, more preferentially less than 2500 ppm, in particular less than 1000 ppm on the basis of the total weight of said composition A. When it is contained in the composition, the total weight content of 2-chloro-1,1,1-trifluoroethane is greater than 1 ppm, advantageously greater than 5 ppm, preferably greater than 10 ppm, more preferentially greater than 20 ppm, in particular greater than 50 ppm, more particularly greater than 100 ppm on the basis of the total weight of said composition A.

According to a preferred embodiment, the composition A comprises 1,2-dichlorohexafluorocyclobutane. 1,2-dichlorohexafluorocyclobutane can exist in the form of two diastereoisomers. The term “1,2-dichlorohexafluorocyclobutane” refers to the two diastereoisomers. Preferably, the total weight content of 1,2-dichlorohexafluorocyclobutane is less than 15%, advantageously less than 10%, preferably less than 5%, in particular less than 1% on the basis of the total weight of said composition A. According to a favored embodiment, the total weight content of 1,2-dichlorohexafluorocyclobutane is less than 5000 ppm, advantageously less than 1000 ppm, preferably less than 500 ppm, more preferentially less than 250 ppm, in particular less than 100 ppm on the basis of the total weight of said composition A. When it is contained in the composition, the total weight content of 1,2-dichlorohexafluorocyclobutane is greater than 1 ppm, advantageously greater than 5 ppm, preferably greater than 10 ppm, more preferentially greater than 20 ppm on the basis of the total weight of said composition A.

According to a preferred embodiment, said composition A comprises at least 80% by weight of chlorotrifluoroethylene on the basis of the total weight of said composition A, advantageously at least 82% by weight, preferably at least 84% by weight, more preferentially at least 86% by weight, in particular at least 88% by weight, more particularly at least 90%, preferably at least 92% by weight of chlorotrifluoroethylene on the basis of the total weight of said composition A.

Said composition A may also comprise trifluoroethylene, preferably in a weight content of less than 5%, preferably less than 4.5%, in particular less than 4% on the basis of the total weight of said composition A.

Said composition A may optionally comprise at least one of the additional compounds C2 chosen from the group consisting of 1,1,2-trifluoroethane, 1-chloro-1,1,2-trifluoroethane, 1-chloro-2,2-difluoroethylene, E/Z-1-chloro-1,2-difluoroethylene and 1-chloro-1,2,2-trifluoroethane. The weight content of said at least one of the additional compounds C2 may be less than 5% on the basis of the total weight of said composition A, advantageously less than 4%, preferably less than 3%, more preferentially less than 2%, in particular less than 1% on the basis of the total weight of said composition A.

### Treatment of the Reaction Flow

The stream B resulting from step a) can be treated in order to recover a purified trifluoroethylene (HFO-1123) stream. Said stream B may comprise, in addition to the trifluoroethylene, HF, HCl, unreacted hydrogen, unreacted chlorotrifluoroethylene, optionally one or more of the additional compounds C1 or C2.

Said stream B may be treated according to the following steps:
i) removal of HF and/or HCl from said product flow obtained in step a) in order to form a gas mixture;
ii) drying of the gas mixture resulting from step i);
iii) treatment of the gas mixture dried in step ii) in order to remove the hydrogen and optionally inert gases;
iv) distillation of the mixture resulting from step iii).

The stream B resulting from step a) is recovered at the reactor outlet in gaseous form. Preferably, at the outlet of the hydrogenolysis reactor, the product flow is first of all treated in order to remove HCl and HF. The product flow is passed through water in a washing column, followed by washing with a dilute base, such as NaOH or KOH. The remainder of the gas mixture, consisting of the unconverted reactants (H₂ and CTFE), dilution nitrogen (if present), trifluoroethylene and additional compounds mentioned above, is directed to a dryer in order to remove the traces of washing water. Drying can be carried out using products such as calcium, sodium or magnesium sulfate, calcium chloride, potassium carbonate, silica gel or zeolites. In one embodiment, a molecular sieve (zeolite), such as siliporite, is used for the drying. The gas mixture, thus dried, is subjected to a step of separation of the hydrogen and inert substances from the remainder of the other products present in the gas mixture by absorption/desorption in the presence of an alcohol comprising from 1 to 4 carbon atoms and preferably ethanol, at atmospheric pressure and at a temperature below ambient temperature, preferably of less than 10° C. and more preferably still at a temperature of −25° C., for the absorption. In one embodiment, the absorption of the organic substances is carried out in a countercurrent column with ethanol cooled to −25° C. The ethanol flow rate is adjusted according to the flow rate of organic substances to be absorbed. The hydrogen and inert gases, which are insoluble in ethanol at this temperature, are removed at the absorption column top. The organic substances are subsequently recovered by heating the ethanol to its boiling point (desorption), in order to be subsequently distilled. Alternatively, step iii) may be carried out by means of a membrane separation process.

According to step iv), the organic compounds thus obtained are distilled to form and recover a stream D1 comprising trifluoroethylene and a stream D2 comprising chlorotrifluoroethylene and optionally one or more of the additional compounds C1 or C2. The stream D2 can be recycled into step a).

According to a preferred embodiment, the distillation step iv) is carried out at a pressure of less than 3 bara, preferably at a pressure of between 0.5 and 3 bara, in particular at a pressure of between 0.9 and 2 bara. Carrying out a distillation at a pressure of less than 3 bara makes it possible to render the process more secure due to the explosive nature of trifluoroethylene above 3 bara. Preferably, the distillation step iv) is carried out in a distillation column comprising a structured packing. It has been observed that a structured packing makes it possible to obtain a more efficient distillation step. The structured packing can be made of a metallic material. Said stream D1 is preferably recovered at the top of the distillation column. Before being recovered, the stream D1 can optionally be partially condensed at the top of the distillation column. When the partial condensation is carried out, the stream D1 is brought to a temperature of −50° C. to −70° C. The temperature is adjusted according to the pressure applied. The partial condensation makes it possible to improve the efficiency of the distillation by limiting the content of additional compounds in the stream D1. Said stream D1 can comprise at least 95% of trifluoroethylene, advantageously at least 96%, preferably at least 97%, in particular at least 98%, more particularly at least 99%, by weight, on the basis of the total weight of said stream B.

### Composition

According to a second aspect, the present invention provides compositions comprising chlorotrifluoroethylene.

Said composition comprises at least 80% by weight of chlorotrifluoroethylene and at least one of the additional compounds chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane; the total weight content of said at least one of the additional compounds is less than 15% on the basis of the total weight of said composition.

According to a preferred embodiment, the composition comprises 1,1,1-trifluoroethane and the total weight content of 1,1,1-trifluoroethane is less than 5000 ppm, advantageously less than 2500 ppm, preferably less than 1000 ppm, more preferentially less than 750 ppm on the basis of the total weight of said composition. When it is contained in the composition, the total weight content of 1,1,1-trifluoroethane is greater than 1 ppm, advantageously greater than 5 ppm, preferably greater than 10 ppm, more preferentially greater than 20 ppm, in particular greater than 50 ppm, more particularly greater than 100 ppm on the basis of the total weight of said composition.

According to a preferred embodiment, the composition comprises 1,1,1,2-tetrafluoroethane and the total weight content of 1,1,1,2-tetrafluoroethane is less than 1000 ppm, advantageously less than 750 ppm, preferably less than 500 ppm, more preferentially less than 250 ppm, in particular less than 100 ppm on the basis of the total weight of said composition. When it is contained in the composition, the total weight content of 1,1,1,2-tetrafluoroethane is greater than 1 ppm, advantageously greater than 5 ppm, preferably greater than 10 ppm, more preferentially greater than 20 ppm on the basis of the total weight of said composition.

According to a preferred embodiment, the composition comprises hexafluorocyclobutene and the total weight content of hexafluorocyclobutene is less than 1%, advantageously less than 7500 ppm, preferably less than 5000 ppm, more preferentially less than 2500 ppm, in particular less than 1000 ppm on the basis of the total weight of said composition. When it is contained in the composition, the total weight content of hexafluorocyclobutene is greater than 1 ppm, advantageously greater than 5 ppm, preferably greater than 10 ppm, more preferentially greater than 20 pm, in particular greater than 50 ppm, more particularly greater than 100 ppm on the basis of the total weight of said composition.

According to a preferred embodiment, the composition comprises fluoroethane and the total weight content of fluoroethane is less than 100 ppm, advantageously less than 75 ppm, preferably less than 50 ppm, more preferentially less than 25 ppm, in particular less than 10 ppm on the basis of the total weight of said composition. When it is contained in the composition, the total weight content of fluoroethane is greater than 0.1 ppm, advantageously greater than 0.5 ppm, preferably greater than 1 ppm on the basis of the total weight of said composition.

According to a preferred embodiment, the composition comprises 2-chloro-1,1,1-trifluoroethane and the total weight content of 2-chloro-1,1,1-trifluoroethane is less than 1%, advantageously less than 7500 ppm, preferably less than 5000 ppm, more preferentially less than 2500 ppm, in particular less than 1000 ppm on the basis of the total weight of said composition. When it is contained in the composition, the total weight content of 2-chloro-1,1,1-trifluoroethane is greater than 1 ppm, advantageously greater than 5 ppm, preferably greater than 10 ppm, more preferentially greater than 20 ppm, in particular greater than 50 ppm, more particularly greater than 100 ppm on the basis of the total weight of said composition.

According to a preferred embodiment, the composition comprises 1,2-dichlorohexafluorocyclobutane and the total weight content of 1,2-dichlorohexafluorocyclobutane is less than 15%, advantageously less than 10%, preferably less than 5%, in particular less than 1% on the basis of the total weight of said composition. According to a favored embodiment, the total weight content of 1,2-dichlorohexafluorocyclobutane is less than 5000 ppm, advantageously less than 1000 ppm, preferably less than 500 ppm, more preferentially less than 250 ppm, in particular less than 100 ppm on the basis of the total weight of said composition. When it is contained in the composition, the total weight content of 1,2-dichlorohexafluorocyclobutane is greater than 1 ppm, advantageously greater than 5 ppm, preferably greater than 10 ppm, more preferentially greater than 20 ppm on the basis of the total weight of said composition.

### EXAMPLE

25 cm³ of catalyst (0.2% of palladium supported on α-alumina) were introduced into a tubular reactor consisting of a stainless steel tube with a length of 1200 mm over a diameter of 25 mm, and equipped with a jacket. The catalyst, thus charged, was subsequently activated in the following way: the reaction tube was placed in a tube furnace and was fed with a stream of hydrogen (from 0.05 to 0.1 mol per gram of catalyst). The catalytic bed is heated to a temperature of 200° C. to 250° C. with a temperature gradient of 0.2° C./min. After this activation period, the tube was cooled to ambient temperature and then was isolated in order to be subsequently installed on a hydrogenolysis test bench.

Four test beds are used in parallel, each comprising a reactor prepared as described above. The four beds were fed with 1 mol/h of starting composition and 1 mol/h of hydrogen in anhydrous form. The temperature of the reactor jacket is 25° C. The contact time, calculated as being the ratio of the volume in liters of catalyst to the sum of the flow rates of the reactants in standard liters per second, was approximately 22 seconds. Tests are carried out using various starting compositions. Comparative example 1 was carried out starting from chlorotrifluoroethylene. Example 2 according to the invention was carried out starting from the chlorotrifluoroethylene used in the comparative example, to which the following compounds were added to obtain a composition A with the proportions mentioned for each of the constituents: 1,1,1-trifluoroethane (519 ppm), 1,1,1,2-tetrafluoroethane (39 ppm), hexafluorocyclobutene (880 ppm), fluoroethane (5 ppm), 2-chloro-1,1,1-trifluoroethane (600 ppm), 1,2-dichlorohexafluorocyclobutane (68 ppm) and trifluoroethylene (2.9%), with the remainder being chlorotrifluoroethylene.

Example 3 according to the invention was carried out starting from the chlorotrifluoroethylene used in the comparative example, to which the following compounds were added to obtain a composition A with the proportions mentioned for each of the constituents: 1,1,1-trifluoroethane (453 ppm), 1,1,1,2-tetrafluoroethane (56 ppm), hexafluorocyclobutene (754 ppm), 2-chloro-1,1,1-trifluoroethane (455 ppm) and 1,2-dichlorohexafluorocyclobutane (54 ppm), with the remainder being chlorotrifluoroethylene.

Example 4 according to the invention was carried out starting from the chlorotrifluoroethylene used in the comparative example, to which the following compounds were added to obtain a composition A with the proportions mentioned for each of the constituents: 1,1,1-trifluoroethane (450 ppm), 1,1,1,2-tetrafluoroethane (52 ppm) and 2-chloro-1,1,1-trifluoroethane (467 ppm), with the remainder being chlorotrifluoroethylene.

The results are shown in table 1 below:

TABLE 1

| | H₂ flow rate | CTFE flow rate | Product VF₃ |
|---|---|---|---|
| Examples | g/h | g/h | g/h |
| Ex. 1 (Comp.) | 2 | 115.87 | 113.04 |
| Ex. 2 (invention) | 2 | 115.87 | 173.33 |
| Ex. 3 (invention) | 2 | 115.87 | 160.67 |
| Ex. 4 (inventive) | 2 | 115.87 | 158.45 |

The productivity mentioned corresponds to the sum of the productivities obtained for all four hydrogenolysis beds. As can be seen, the trifluoroethylene productivity is significantly improved starting from the composition according to the invention compared with a chlorotrifluoroethylene composition without the additional compounds.

1. A process for producing trifluoroethylene in a reactor equipped with a fixed catalytic bed comprising a catalyst, said process comprising a step a) of reacting a composition A comprising chlorotrifluoroethylene with hydrogen in the presence of a catalyst and in the gas phase in order to produce a stream B comprising trifluoroethylene,
wherein said composition A also comprises at least one of the additional compounds C1 chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane.

2. The process as claimed in claim 1, wherein the total weight content of said at least one of the additional compounds C1 is less than 15% on the basis of the total weight of said composition A.

3. The process as claimed in claim 1, wherein said composition A comprises at least 80% by weight of chlorotrifluoroethylene on the basis of the total weight of said composition A.

4. The process as claimed in claim 1, wherein said composition A also comprises trifluoroethylene.

5. The process as claimed in claim 1, wherein said composition A also comprises at least one of the additional compounds C2 chosen from the group consisting of 1,1,2-trifluoroethane, 1-chloro-1,1,2-trifluoroethane, 1-chloro-2,2-difluoroethylene, E/Z-1-chloro-1,2-difluoroethylene and 1-chloro-1,2,2-trifluoroethane.

6. The process as claimed in claim 5, wherein the weight content of said at least one of the additional compounds C2 is less than 5% on the basis of the total weight of said composition A.

7. The process as claimed in claim 1, wherein the catalyst comprises palladium supported on α-alumina.

8. The process as claimed in claim 1, wherein the chlorotrifluoroethylene and the hydrogen are in anhydrous form.

9. The process as claimed in claim 1, wherein said process comprises a step i′) of activating the catalyst, carried out prior to step a), by bringing said catalyst into contact with a gas stream comprising a reducing agent, an inert gas or a mixture thereof.

10. The process as claimed in claim 9, wherein, during said step i′):
the temperature of the catalytic bed is increased from a temperature T1 to a temperature T2 that is greater than T1, with a temperature gradient of less than 0.5° C./min; or
the temperature of the catalytic bed is increased from a temperature T1 to a temperature T2 that is greater than T1, in increments.

11. A composition comprising at least 80% by weight of chlorotrifluoroethylene and at least one of the additional compounds chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane; the total weight content of said at least one of the additional compounds is less than 15% on the basis of the total weight of said composition.

12. The composition as claimed in claim 11, wherein the composition comprises at least two of the additional compounds chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane.

13. The composition as claimed in claim 11, wherein the composition comprises at least three of the additional compounds chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane.

14. The composition as claimed in claim 11, wherein the composition comprises 1,1,1-trifluoroethane, 1,1,1,2-tetrafluoroethane, and 2-chloro-1,1,1-trifluoroethane.

15. The composition as claimed in claim 11, wherein the composition comprises at least one of the additional compounds chosen from the group consisting of
at least 50 ppm of 1,1,1-trifluoroethane,
at least 10 ppm of 1,1,1,2-tetrafluoroethane,
at least 50 ppm of hexafluorocyclobutene,
at least 1 ppm of fluoroethane,
at least 50 ppm of 2-chloro-1,1,1-trifluoroethane, and
at least 10 ppm of 1,2-dichlorohexafluorocyclobutane.

16. The process as claimed in claim 1, wherein said composition A comprises at least two of the additional compounds C1 chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane.

17. The process as claimed in claim 1, wherein said composition A comprises at least three of the additional compounds C1 chosen from the group consisting of 1,1,1-trifluoroethane, 1,1,1,2-tetrafluoroethane, hexafluorocyclobutene, fluoroethane, 2-chloro-1,1,1-trifluoroethane and 1,2-dichlorohexafluorocyclobutane.

18. The process as claimed in claim 1, wherein said composition A comprises 1,1,1-trifluoroethane, 1,1,1,2-tetrafluoroethane, and 2-chloro-1,1,1-trifluoroethane.

19. The process as claimed in claim 1, wherein said composition A comprises at least one of the additional compounds C1 chosen from the group consisting of
at least 50 ppm of 1,1,1-trifluoroethane,
at least 10 ppm of 1,1,1,2-tetrafluoroethane,
at least 50 ppm of hexafluorocyclobutene,
at least 1 ppm of fluoroethane,
at least 50 ppm of 2-chloro-1,1,1-trifluoroethane, and
at least 10 ppm of 1,2-dichlorohexafluorocyclobutane.
